(19) United States
(12) Patent Application Publication
(10) Pub. No.: US 2008/0051729 A1
(43) Pub. Date: Feb. 28, 2008

(54) Needle syringe having some disposable components

(75) Inventor: Wan Chang Cheng, Chu San Cheng (TW)

Correspondence Address: Cheng Wan Chang, 235 Chung-Ho Box 8-24, Taipei, omitted

(73) Assignees: Hsien Tsung WANG; Shin-Lin LIANG

(21) Appl. No.: 11/647,225

(22) Filed: Dec. 29, 2006

(30) Foreign Application Priority Data
Aug. 24, 2006 (CN) 200610111919.3

(51) Int. Cl. A61M 5/00

(52) U.S. Cl. 604/232

(57) ABSTRACT

A needle syringe in one embodiment includes a needle assembly including front and rear hypodermic needles; a barrel including a first window; a tube threadedly secured to the barrel and the needle assembly respectively and including a second window and an inner circumferential tapered surface; a medicine cartridge including a rear plunger and a front plug secured to the tapered surface when the cartridge is mounted in the barrel; and a handle. The rear needle passes the tube and the plug into the cartridge. The cartridge is prohibited from slipping relative to the barrel during injection. A medical worker can view the volume of fluid in the cartridge through the windows so as to correctly determine when to stop injection. After use the tube, the cartridge, and the needle assembly are removed to be discarded, and the barrel, the wing, and the handle are adapted to re-use.

### BACKGROUND OF THE INVENTION

1. Field of Invention

The present invention relates to needle syringes and more particularly to such a needle syringe which after use and after a simple disassembly some components including a tube, a medicine cartridge, and a double needle are discarded as a whole for reducing the risk of spreading blood-borne diseases, and the remaining components including a barrel, a wing, and a handle assembly adapted to re-use by sterilization for being cost-effective.

2. Description of Related Art

A conventional needle syringe is shown in FIGS. 1, 2, and 3. The needle syringe comprises a barrel 90, a wing 91, and a pivot 92 for pivotably securing a rear end of the barrel 90 to the wing 91. As such, the wing 91 is adapted to turn about 90-degree relative to the barrel 90 for mounting an anesthetic cartridge 93 in the barrel 90. The barrel 90 comprises a detachable head 94 having interior threads adapted to threadably secure to outer threads on a forward end of the barrel 90. A needle housing 95 having two opposite hypodermic needles 951 and 952 (called double needle 95 hereinafter) extended therefrom is threadably secured to outer threads on a forward end of the head 94. The rear needle 951 is adapted to pierce into the anesthetic cartridge 93 for sucking liquid therefrom. A lengthwise window 96 is formed on a surface of the barrel 90. A medical worker may view the volume of fluid in the anesthetic cartridge 93 through the window 96. After injection, the double needle 95, the head 94, and the anesthetic cartridge 93 are removed sequentially. Both the double needle 95 and the anesthetic cartridge 93 are adapted to dispose (i.e., discarded). The barrel 90, the wing 91, and the head 94 are made of stainless steel. Thus, the barrel 90, the wing 91, and the head 94 are relatively expensive in consideration of cost and they are sterilized for re-use. However, a patient may be contaminated by the re-used needle syringe if the sterilization is not complete. Moreover, the double needle 95 may accidentally prick the medical worker with microorganisms while removing.

Referring to FIG. 3 specifically, a medical worker may press a handle 911 to push a rear plunger 931 of the anesthetic cartridge 93. Hence, fluid is expelled out of the anesthetic cartridge 93 and is then injected into the body cavities of a patient through the double needle 95. It is typically of turning the handle 911 to cause a forward helical hook 912 thereof to thread into the plunger 931 prior to the injection. However, it is often that the anesthetic cartridge 93 may turn the same as the handle 931 turns. That is, the anesthetic cartridge 93 may slip relative to the barrel 90. This can increase difficulties of piercing the hook 912 into the plunger 931. Typically, a great force is exerted upon the handle 911 for achieving the piercing. This is not desirable. Moreover, an enhancement 901 is formed around a forward portion of the barrel 90 for increasing a structural strength of the barrel 90 and facilitating a medical worker to hold the barrel 90. However, the provision of the enhancement 901 partially hinders the field of view of the medical worker when observing the position of the plunger 931 in the anesthetic cartridge 93 and the remaining fluid in the anesthetic cartridge 93 during the injection. As a result, a correct determination of when to stop the injection is made impossible.

In addition, many needle syringes are made of plastic nowadays. These needle syringes are typically disposable for reducing the risk of spreading blood-borne diseases. However, discarding a great number of needle syringes after use is not cost-effective and may further pollute the environment if they are not disposed properly. Thus, the need for improvement still exists.

### SUMMARY OF THE INVENTION

It is therefore one object of the present invention to provide a needle syringe having windows formed on a surface of its barrel for enabling a medical worker to clearly view the volume of fluid in a medicine cartridge through windows during injection so as to make a correct determination of when to stop the injection.

It is another object of the present invention to provide a needle syringe which after use and after a simple disassembly some components including a tube, a medicine cartridge, and a double needle thereof are discarded as a whole for reducing the risk of spreading blood-borne diseases, and the remaining components including a barrel, a wing, and a handle assembly adapted to re-use for being cost-effective.

It is a further object of the present invention to provide a needle syringe including a tube having a plurality of equally spaced lengthwise ribs around its inner surface so as to form a tapered surface or tapered surface inner barrel for fastening a forward plug of a medicine cartridge mounted in a barrel. Thus, the medicine cartridge is prohibited from slipping relative to the barrel when a plunger is sliding in the medicine cartridge toward the plug for dispensing fluid.

In one aspect of the present invention there is provided a needle syringe comprising a wing; a needle assembly including a front hypodermic needle and a rear hypodermic needle being in communication with each other; a barrel including a lengthwise first window formed on a surface, and a pivot for pivotably securing the wing to a rear end of the barrel; a tube threadedly secured to a forward end of the barrel and the needle assembly respectively with the rear needle disposed in the tube, the tube including a lengthwise second window formed on a surface, and a plurality of equally spaced lengthwise internal ribs formed between the second window and a forward end; a medicine cartridge including a rear sliding plunger and a front plug secured to the ribs when the medicine cartridge is mounted in the barrel through the rear end of the barrel, wherein the rear needle pierces through the plug into the medicine cartridge; and a handle including a forward pointed member secured to the plunger, wherein the handle is adapted to push to slide the plunger forward to expel fluid in the medicine cartridge out of the front needle through the rear needle; and after use the tube, the medicine cartridge, and the needle assembly are removed to be discarded, and the barrel, the wing, and the handle are adapted to re-use.

In another aspect of the present invention the tube further comprises an upper projection formed on the surface proximate the second window, and there is further provided a sleeve including a lower, internal longitudinal trough and a longitudinal slot including a protrusion proximate a forward end, a cavity at a bottom end, a lower indentation, and a flexible ratchet tooth between the cavity and the indentation, wherein in an assembly, the projection is fastened in the indentation after inserting into the trough and turning a predetermined angle with the needle assembly concealed in the sleeve; wherein either the tube or the sleeve is adapted to turn to move the projection to be aligned with the slot such that pushing the plunger will slide the projection along the slot until passing the protrusion and being fastened between the forward end of the slot and the protrusion for expelling fluid in the medicine cartridge out of the front needle through the rear needle; and wherein after use pulling the plunger will slide the projection along the slot until passing the indentation and the ratchet tooth to be locked in the cavity such that the medicine cartridge the tube, the needle assembly, and the sleeve are adapted to remove to be discarded.

In a further aspect of the present invention there is provided a needle syringe comprising a wing; a needle assembly including a front hypodermic needle and a rear hypodermic needles being in communication with each other; a barrel threadedly secured to the wing and including a lengthwise first window formed on a surface; a tube threadedly secured to a forward end of the barrel and the needle assembly respectively with the rear needled disposed in the tube, the tube including an inner tapered receptacle formed in an inner surface of the transparent tube in the rear of a forward end; a medicine cartridge including a rear sliding plunger and a front plug secured to the receptacle when the medicine cartridge is mounted in the barrel through the first window wherein the rear needle pierces through the plug into the medicine cartridge; and a handle including a forward pointed member secured to the plunger, wherein the handle is adapted to push to slide the plunger forward to expel fluid in the medicine cartridge out of the front needle through the rear needle; and after use the tube, the medicine cartridge, and the needle assembly are removed to be discarded, and the barrel, the wing, and the handle are adapted to re-use.

The above and other objects, features and advantages of the invention will become apparent from the following detailed description taken with the accompanying drawings.

### BRIEF DESCRIPTION OF THE DRAWINGS

FIG. 1 is a perspective view of a conventional needle syringe;

FIG. 2 is an exploded perspective view of FIG. 1;

FIG. 3 is a longitudinal sectional view of the main portion of the needle syringe of FIG. 1;

FIG. 4 is a perspective view of a first preferred embodiment of needle syringe according to the invention;

FIG. 5 is an exploded perspective view of FIG. 4;

FIG. 6 is a longitudinal sectional view of the main portion of the needle syringe of FIG.4;

FIG. 7 is an exploded perspective view of FIG. 4 showing the joined transparent tube, medicine cartridge, and double needle being detached to be discarded for disposal after injection;

FIG. 8 is an exploded perspective view of a second preferred embodiment of needle syringe according to the invention;

FIG. 9 is an exploded perspective view of a third preferred embodiment of needle syringe according to the invention;

FIG. 10 is a longitudinal sectional view of main portion of the needle syringe of FIG. 9;

FIG. 10A is a detailed view of the area in a circle of FIG. 10;

FIG. 11 an exploded perspective view of a fourth preferred embodiment of needle syringe according to the invention;

FIG. 12 is a schematic longitudinal sectional view of the main portion of the needle syringe of FIG. 11 with the sleeve put on both the barrel and the transparent tube and the needle projecting out of a top opening of the sleeve for injection;

FIG. 13 is a view similar to FIG. 12 where the barrel has been pulled downward to completely lockingly conceal the needle in the sleeve after the injection; and

FIG. 14 is an exploded perspective view of the needle syringe of FIG. 11 where the medicine cartridge, the transparent tube, the double needle, and the sleeve are joined together prior to disposal.

### DETAILED DESCRIPTION OF THE INVENTION

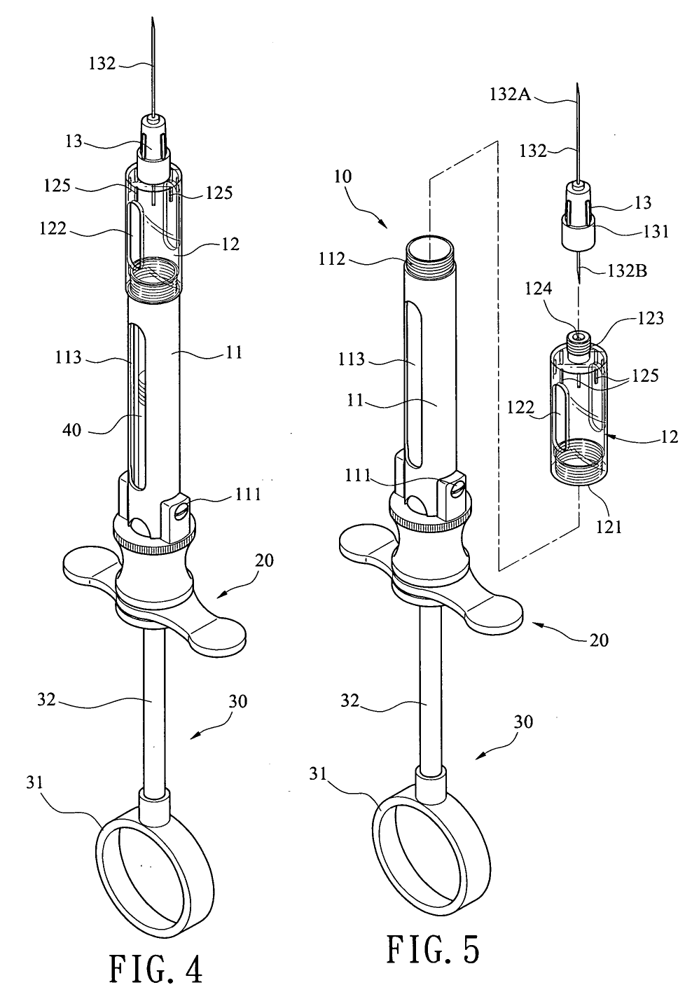

Referring to FIGS. 4 to 7, a needle syringe in accordance with a first preferred embodiment of the invention is shown. The needle syringe comprises a barrel and needle assembly 10, a wing 20, a handle assembly 30, and a medicine cartridge 40. Each component is discussed in detailed below. The barrel and needle assembly 10 comprises a hollow, cylindrical barrel 11 including a forward externally threaded extension 112, a lengthwise first window 113 formed on a surface of the barrel 11, and a pivot 111 for pivotably securing a rear end of the barrel 11 to the wing 20. As such, the wing 20 is adapted to turn about 90-degree relative to the barrel 11 for mounting the medicine cartridge 40 in the barrel and needle assembly 10 as detailed later.

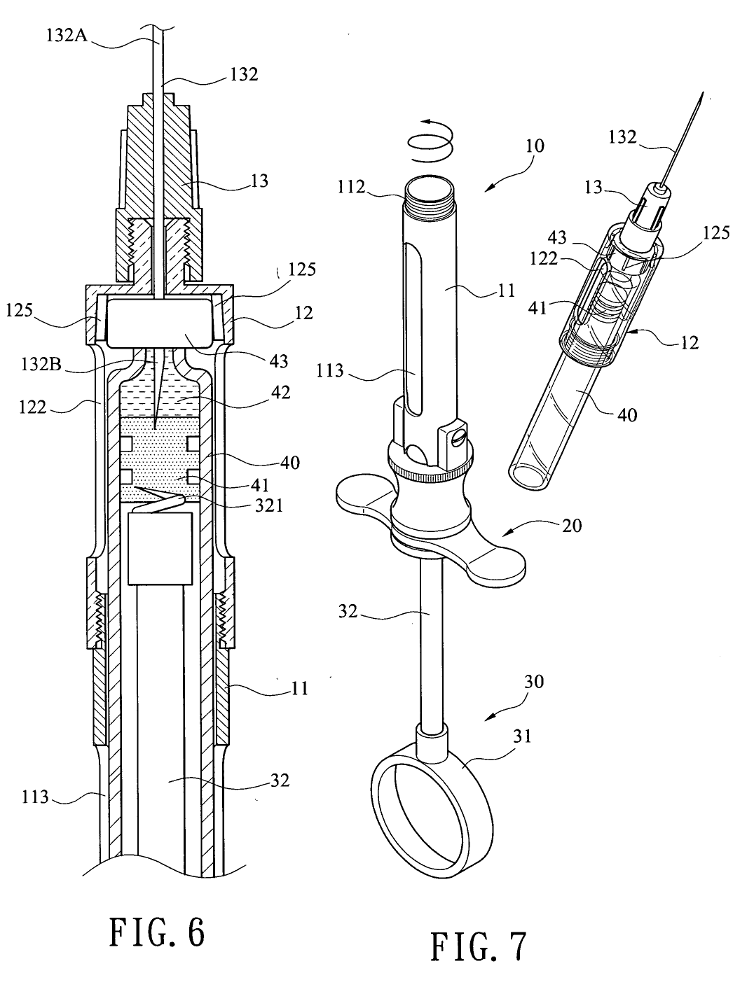

The barrel and needle assembly 10 further comprises a transparent tube 12 including rear internal threads 121, a lengthwise second window 122 formed on a surface of the transparent tube 12, a forward nose 123 having external threads, an opening 124 on an open end of the nose 123, and a plurality of equally spaced lengthwise ribs 125 formed around an inner surface of the transparent tube 12 between the second window 122 and the nose 123. The ribs 125 are adapted to form a truncated conic member having a diameter gradually decreased toward its front ends. An inner diameter measured at rear ends of two opposite ribs 125 is larger than that measured at front ends thereof, i.e., an inner tapered surface is formed by the ribs 125 as shown in FIG. 6.

The barrel and needle assembly 10 further comprises a needle housing (called double needle hereinafter) 13 including a casing 131 and a needle unit 132 including two hypodermic needles 132A and 132B extended oppositely from the casing 131 and being in fluid communication each other. The casing 131 comprises rear internal threads (not shown).

The cylindrical medicine cartridge 40 comprises a rear plunger 41, a front soft plug 43, and fluid (e.g., anesthetic) 42 sealed between the plunger 41 and the plug 43.

The handle assembly 30 comprises a rear ring-shaped handle 31 and an arm 32.

In assembly, pivot the wing 20 about 90-degree relative to the barrel 11. Next, insert the medicine cartridge 40 into the barrel 11 from its open rear end. Next, pivot the wing 20 in an opposite direction to close the rear end of the barrel 111 so as to mount the medicine cartridge 40 in the barrel 11. Next, threadably secure the internal threads 121 to the extension 112 for mounting the transparent tube 12 and the barrel 11 together with the plug 43 being secured to the inner tapered surface formed by the ribs 125. Next, threadably drive the casing 131 onto the nose 123 while piercing the rear needle 132B through the plug 43 into the medicine cartridge 40 until the casing 131 and the nose 123 are secured together, i.e., the double needle 13 and the transparent tube 12 mounted together. Finally, push the arm 32 to cause a forward helical hook 321 thereof to thread into the plunger 41 until they are secured together.

For dispensing the fluid 42, a medical worker may push the handle assembly 30 forward by holding the handle 31 to cause the arm 32 to push the plunger 41 forward. Advantageously, the medicine cartridge 40 is prohibited from slipping relative to the barrel 11 because the plug 43 is secured to the inner tapered surface formed by the plurality of ribs 125. As such, the plunger 41 is adapted to smoothly slide within the medicine cartridge 40 toward the plug 43 while the fluid 42 is expelling out of the needle syringe through the needle unit 132 into the body cavities of a patient. The medical worker may view the volume of fluid 42 in the medicine cartridge 40 through both the windows 113 and 122 clearly during the injection. This can effect an optimum injection. Alternatively, the medical personal can stop the injection once the pushing is stopped due to the secure engagement of the plunger 41 with the sharp end of the needle 132B by piercing. Therefore, a correct determination of when to stop the injection is made possible.

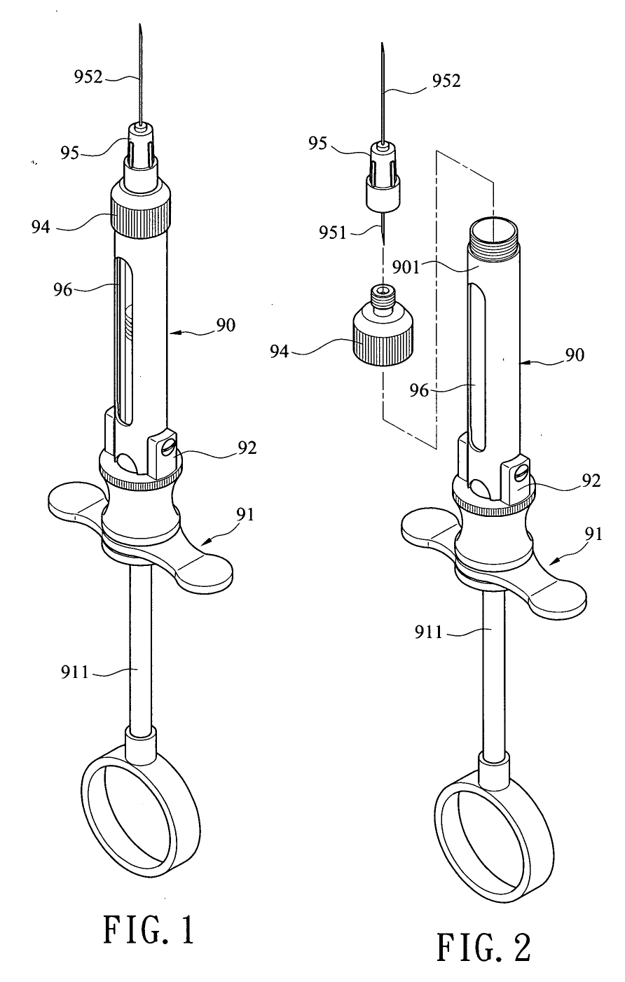

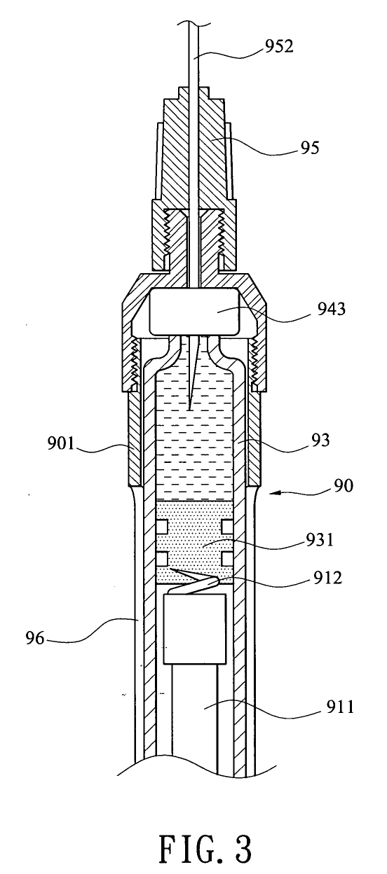

In the invention the barrel 11, the wing 20, and the handle assembly 30 are made of stainless steel. Referring to FIG. 7 specifically, after use the barrel 11, the wing 20, and the handle assembly 30 are detached from the remaining components of the needle syringe by unfastening the barrel 11 and the transparent tube 12. Thereafter, the barrel 11, the wing 20, and the handle assembly 30 can be re-used by sterilization. This can save cost. Further, the transparent tube 12, the medicine cartridge 40, and the double needle 13 are discarded as a whole for reducing the risk of spreading blood-borne diseases. As compared to the prior art which involves removing the double needle 95 out of the head 94 by exerting a great force due to the piercing engagement of the rear needle 951 with the anesthetic cartridge 93 (see FIGS. 1 and 2), the invention is not only safer and labor-saving but also is quick and convenient in the removal. All of the above are contemplated by the invention.

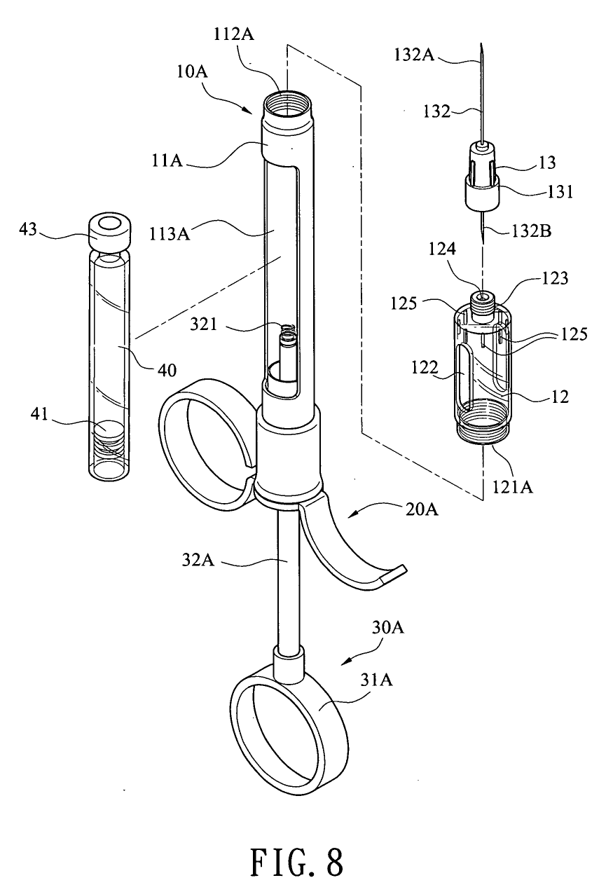

Referring to FIG. 8, a needle syringe in accordance with a second preferred embodiment of the invention is shown. The characteristics of the second preferred embodiment are detailed below. A barrel 11A of needle assembly 10A is threadably secured to a wing 20A. The medicine cartridge 40 is placed in the barrel 11A from a large first window 113A formed on a surface of the barrel 11A. The barrel 11A comprises a forward internally threaded extension 112A. A transparent tube 12 comprises a rearward externally threaded extension 121A adapted to secure to the extension 112A. For brevity, the remaining components same as the above embodiment are designated with same reference numeral, the related descriptions are thereby omitted.

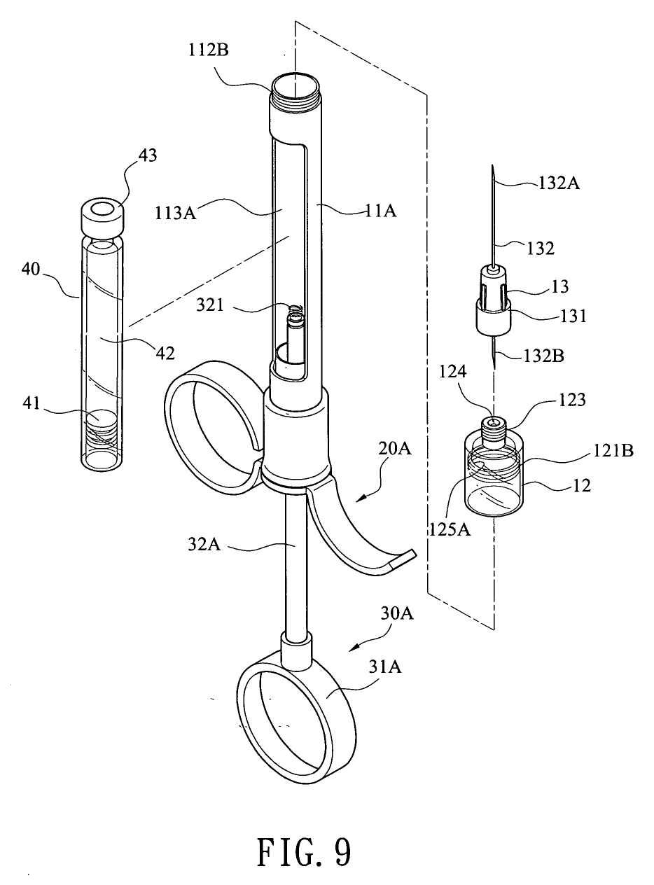

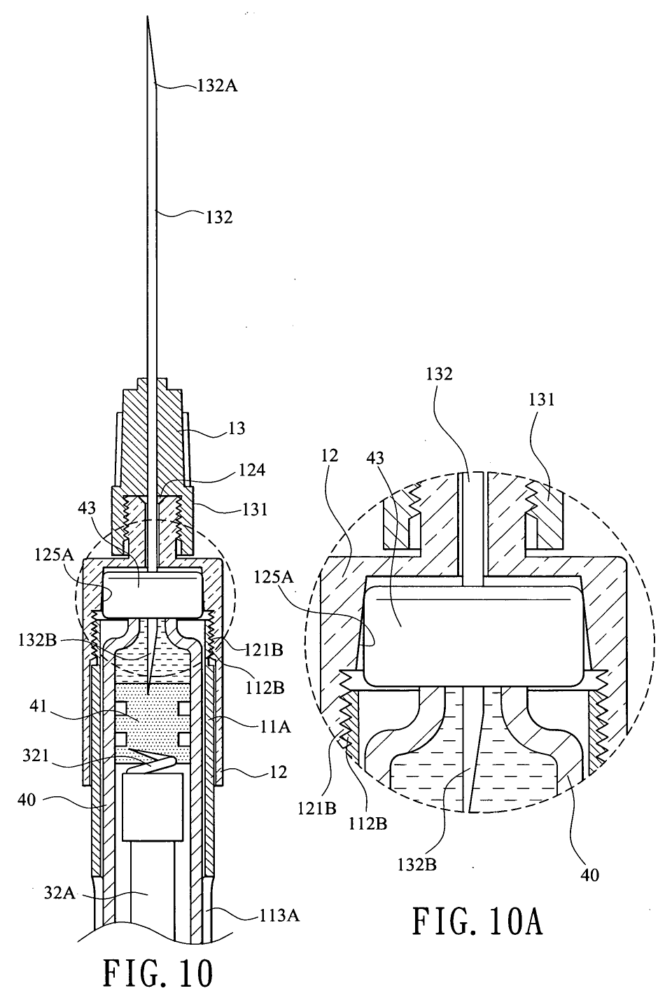

Referring to FIGS. 9, 10 and 10A, a needle syringe in accordance with a third preferred embodiment of the invention is shown. The configuration of the third preferred embodiment of the invention is same as the above second embodiment except the followings. The characteristics of the third preferred embodiment are detailed below. The barrel 11A comprises an externally threaded extension 112B. A transparent tube 12 comprises a middle internally threaded extension 121B adapted to secure to the extension 112B. An inner tapered receptacle 125A formed around an inner surface of the transparent tube 12 in the rear of the nose 123. An inner diameter measured at rear end of the receptacle 125A is larger than that measured at front end of the plug 43 that is the receptacle 125A is adapted to form an inner tapered conic surface having an inner diameter gradually decreased toward a front end thereof. Thus, the plug 43 is secured to the inner tapered surface formed by the receptacle 125A, as shown in FIGS. 10 and 10A. As such, the plunger 41 is adapted to smoothly slide within the medicine cartridge 40 toward the plug 43 while the fluid 42 is expelling out of the needle syringe through the needle unit 132 into the body cavities of a patient. For brevity, the remaining components same as the above embodiment are designated with same reference numerals, the related descriptions are thereby omitted.

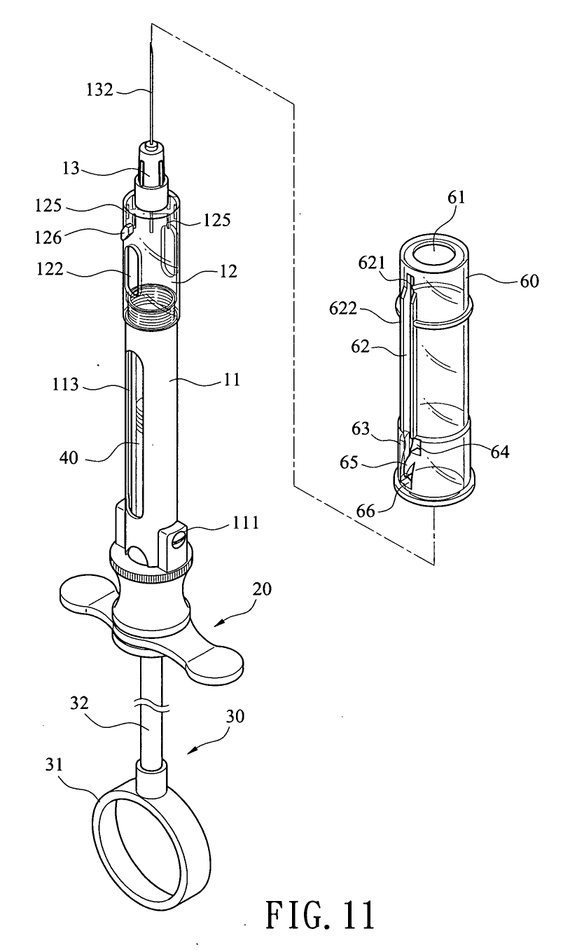

Referring to FIGS. 11, 12, 13, and 14, a needle syringe in accordance with a fourth preferred embodiment of the invention is shown. The configuration of the fourth preferred embodiment of the invention is same as the above second embodiment except the followings. The characteristics of the fourth preferred embodiment are detailed below. The transparent tube 12 includes a projection 126 formed thereon proximate the ribs 125. A sleeve 60 is provided and comprises a top opening 61, a longitudinal slot 62 through almost the entire length of the sleeve 60, and a lower, internal longitudinal trough 63. The slot 62 includes a protrusion 621 proximate a forward end, a bottom cavity 66, a lower side indentation 64, a flexible ratchet tooth 65 between the cavity 66 and the indentation 64, and two parallel flanges 622 along edges of the slot 62 and between the protrusion 621 and the indentation 64. In the assembly, the projection 126 is inserted into the trough 63 and then the projection 126 is turned to enter the indentation 64 for fastening before use. As such, the sleeve 60 and the transparent tube 12 are assembled together with the double needle 13 concealed in the sleeve 60 prior to delivery.

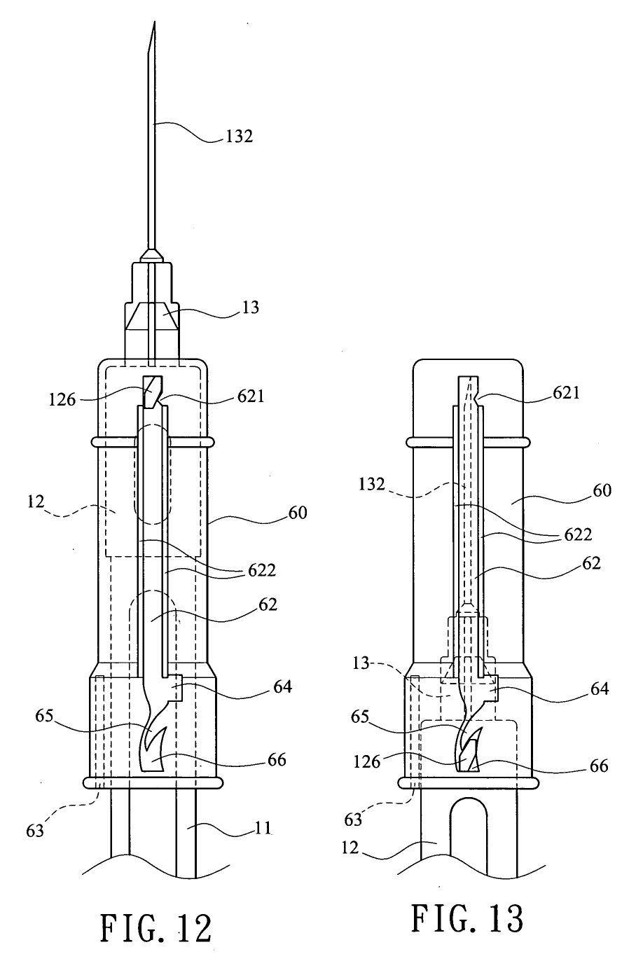

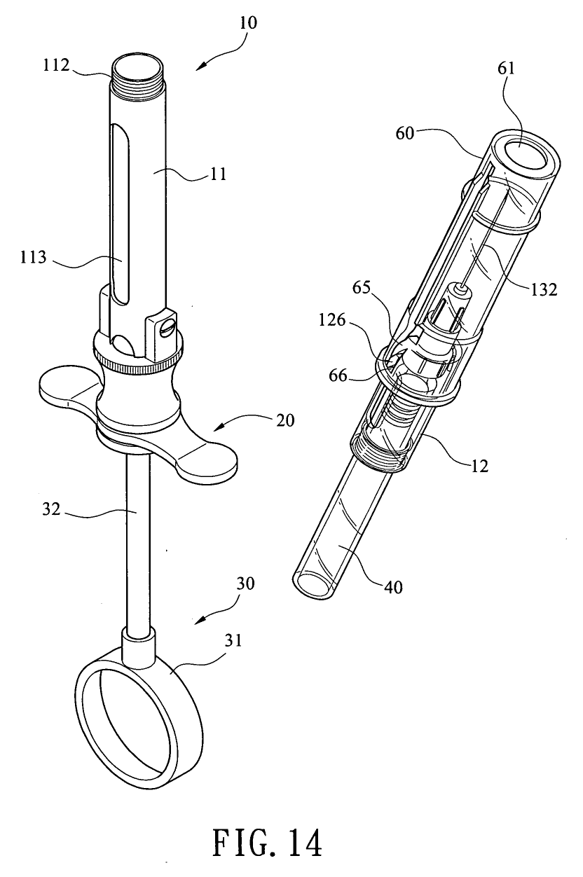

For projecting the double needle 13 out of the top opening 61 of the sleeve 60 prior to dispensing fluid, a medical worker may turn the transparent tube 12 or the sleeve 60 to move the projection 126 to be aligned with the slot 62. Next, slide the projection 126 along the slot 62 until passing the protrusion 621 and being fastened between the forward end of the slot 62 and the protrusion 621 (see FIG. 12). Thereafter, the medical worker can press the handle assembly 30 to inject the fluid through the needle unit 132 into the body cavities of a patient. After the injection, the medical worker may slide the projection 126 downward along the slot 62 until passing the indentation 64 and the ratchet tooth 65 to be locked in the cavity 66 by the ratchet tooth 65 (see FIG. 13). At this position, the double needle 13 is completely lockingly concealed in the sleeve 60. Thus, the medicine cartridge 40 the transparent tube 12, the double needle 13, and the sleeve 60 are lockingly joined together and can be discarded as a whole (see FIG. 14). Likewise, other remaining components such as the barrel 11, the wing 20, and the handle assembly 30 can be re-used by sterilization for being cost-effective.

While the invention herein disclosed has been described by means of specific embodiments, numerous modifications and variations could be made thereto by those skilled in the art without departing from the scope and spirit of the invention set forth in the claims.

What is claimed is:

1. A needle syringe comprising:
a wing;
a needle assembly including a front hypodermic needle and a rear hypodermic needle being in communication with each other;
a barrel including a lengthwise first window formed on a surface, and a pivot for pivotably securing the wing to a rear end of the barrel;
a tube threadedly secured to a forward end of the barrel and the needle assembly respectively with the rear needle disposed in the tube, the tube including a lengthwise second window formed on a surface, and a plurality of equally spaced lengthwise internal ribs formed between the second window and a forward end;
a medicine cartridge including a rear sliding plunger and a front plug secured to the ribs when the medicine cartridge is mounted in the barrel through the rear end of the barrel, wherein the rear needle pierces through the plug into the medicine cartridge; and
a handle including a forward pointed member secured to the plunger, wherein:
the handle is adapted to push to slide the plunger forward to expel fluid in the medicine cartridge out of the front needle through the rear needle; and
after use the tube, the medicine cartridge, and the needle assembly are removed to be discarded, and the barrel, the wing, and the handle are adapted to be re-used.

2. The needle syringe of claim 1, wherein the tube is transparent.

3. The needle syringe of claim 1, wherein the tube is opaque.

4. The needle syringe of claim 1, wherein the barrel, the wing, and the handle are made of stainless steel.

5. The needle syringe of claim 1, wherein the ribs are adapted to form a truncated conic member having a diameter gradually decreased toward front ends thereof.

6. The needle syringe of claim 1, wherein the tube further comprises an upper projection formed on the surface proximate the second window, and further comprising a sleeve including a lower, internal longitudinal trough and a longitudinal slot including a protrusion proximate a forward end, a cavity at a bottom end, a lower indentation, and a flexible ratchet tooth between the cavity and the indentation, wherein in an assembly, the projection is fastened in the indentation after inserting into the trough and turning a predetermined angle with the needle assembly concealed in the sleeve; wherein either the tube or the sleeve is adapted to turn to move the projection to be aligned with the slot such that pushing the plunger will slide the projection along the slot until passing the protrusion and being fastened between the forward end of the slot and the protrusion for expelling fluid in the medicine cartridge out of the front needle through the rear needle; and wherein after use pulling the plunger will slide the projection along the slot until passing the indentation and the ratchet tooth to be locked in the cavity such that the medicine cartridge the tube, the needle assembly, and the sleeve are adapted to remove to be discarded.

7. A needle syringe comprising:
a wing;
a needle assembly including a front hypodermic needle and a rear hypodermic needles being in communication with each other;
a barrel threadedly secured to the wing and including a lengthwise first window formed on a surface;
a tube threadedly secured to a forward end of the barrel and the needle assembly respectively with the rear needled disposed in the tube, the tube including an inner tapered receptacle formed in an inner surface of the transparent tube in the rear of a forward end thereof;
a medicine cartridge including a rear sliding plunger and a front plug secured to the receptacle when the medicine cartridge is mounted in the barrel through the first window wherein the rear needle pierces through the plug into the medicine cartridge; and
a handle including a forward pointed member secured to the plunger, wherein:
the handle is adapted to push to slide the plunger forward to expel fluid in the medicine cartridge out of the front needle through the rear needle; and
after use the tube, the medicine cartridge, and the needle assembly are removed to be discarded, and the barrel, the wing, and the handle are adapted to bere-used.

8. The needle syringe of claim 7, wherein the tube is transparent.

9. The needle syringe of claim 7, wherein the tube is opaque.

10. The needle syringe of claim 7, wherein the barrel, the wing, and the handle are made of stainless steel.

11. The needle syringe of claim 7, wherein the receptacle is adapted to form an inner tapered conic member having an inner diameter gradually decreased toward a front end thereof.

12. The needle syringe of claim 7, wherein the tube further comprises an upper projection formed on the surface proximate the second window, and further comprising a sleeve including a lower, internal longitudinal trough and a longitudinal slot including a protrusion proximate a forward end, a cavity at a bottom end, a lower indentation, and a flexible ratchet tooth between the cavity and the indentation, wherein in an assembly, the projection is fastened in the indentation after inserting into the trough and turning a predetermined angle with the needle assembly concealed in the sleeve; wherein either the tube or the sleeve is adapted to turn to move the projection to be aligned with the slot such that pushing the plunger will slide the projection along the slot until passing the protrusion and being fastened between the forward end of the slot and the protrusion for expelling fluid in the medicine cartridge out of the front needle through the rear needle; and wherein after use pulling the plunger will slide the projection along the slot until passing the indentation and the ratchet tooth to be locked in the cavity such that the medicine cartridge the tube, the needle assembly, and the sleeve are adapted to remove to be discarded.
